(19) United States
(12) Patent Application Publication
(10) Pub. No.: US 2009/0181974 A1
(43) Pub. Date: Jul. 16, 2009

(54) Composition for slowing down the development of alzheimer's disease

(76) Inventors: Dominique Bourgeade, Lyon (FR); Jean-Christophe Anton, Strasbourg (FR); Francois Miceli, Lyon (FR)

Correspondence Address: Blakely Sokoloff Taylor & Zafman, 1279 Oakmead Parkway, Sunnyvale, CA, 94085-4040, US

(21) Appl. No.: 11/921,066

(22) PCT Filed: May 24, 2006
(86) PCT No.: PCT/EP2006/062608

§ 371 Date: Feb. 13, 2009

(30) Foreign Application Priority Data
May 27, 2005 (FR) 0505365

(51) Int. Cl. A61K 31/519; A61P 25/28

(52) U.S. Cl. 514/249

(57) ABSTRACT

The invention relates to a composition comprising: vegetable oil containing at least one linoleic acid and at least one linolenic acid, vitamin E, and vitamin B9 (or folates), whereby the linoleic acid/linolenic acid weight ratio is between 3 and 5. The inventive composition is intended to slow down the development of Alzheimer's disease and to improve the patient's quality of life. Said composition is intended to be incorporated in a vector that can be assimilated by the human body, preferably by means of dietary intake.

The present invention relates to a composition allowing the progression of Alzheimer's disease to be delayed and patients' quality of life to be improved. This composition is intended to be incorporated in a vector which can be absorbed by the human body, preferably via food intake.

The composition of the invention may also be given veterinary use as food supplement for animals, in particular carnivorous domestic animals and more specifically dogs or cats.

Alzheimer's disease is a type of dementia which generally affects elderly persons, for which at the present time no decisive treatment exists. It is characterized by gradual, irreversible deterioration of the cognitive functions and by particular neuro-histological lesions. At the current time, a low number of medicinal products exist which may, but only transiently, improve the condition of some patients suffering from this disease.

This neuro-degenerative disorder of the central nervous system firstly causes a severe amnesia syndrome, which chiefly results from lesions of the neurons of the hippocampus, a converging region for memorized information and the first site in which the disease is located. Thereafter, the lesions extend towards the neo-cortex and more precisely towards the associative areas, thereby leading to disturbed so-called instrumental functions. The lesions associated with the disease are cortical atrophy, senile plaques (deposit of an amyloid substance), and neurofibrillary degeneration.

Several biochemical mechanisms are involved in the physiopathological process of Alzheimer's disease, but their respective roles are currently not clearly established.

The first cognitive signs are generally memory disorders and difficulties in performing activities of daily living. However visual and spatial disorders, and language disorders sometimes complete the picture. The characteristic memory disorders of Alzheimer's disease firstly concern the short-term memory which enables the fixing of new information, leading in particular to time disorientation. Disorientation in space usually comes later. Finally, the patient has increasing difficulty in performing a cognitive task of greater or less complexity.

Alzheimer's also generates non-cognitive ill-functioning such as depression, anxiety, apathy, a certain form of verbal and physical aggressiveness, as well as sleep disorders and hyperphagia.

Having regard to the involvement of the cholinergic system in the cognitive deficiency of patients suffering from the disease, treatments initially gave preference to a concentration of acetylcholine. Nonetheless, no treatment available today is able to halt the progression of the disease or to reverse its course. They simply allow the onset of aggravation symptoms to be delayed.

There is therefore a strong need to improve the slowing of the progression of the disease, which entails acting as early as possible on the precursor signs (biological and physiological) of Alzheimer's, to prevent or delay the onset and progression of neuro-histological lesions whilst preserving or improving patients' quality of life.

The present invention therefore concerns a novel composition which can allow the progression of Alzheimer's disease to be delayed and patients' quality of life to be improved.

Numerous compositions of food supplements containing mixtures of fatty acids, vitamin cocktails and optionally mineral salts, sources of proteins and sugars, are known in the state of the art. However, according to the type of ingredients that are associated, the applications of these food supplements range from “body building” to the treatment of depression and increased breast milk production.

For example patent application US 2003/0580341 describes a combination of vitamins with DHA to treat a multitude of disorders in adults or children, related to an allegedly beneficial effect of DHA.

Patent application US 2004/0071825 describes cocktails of vitamins, minerals and plant extracts in granule form, for “body building” in particular or to increase intellectual activity.

Patent application EP 815 735 describes dairy products for routine consumption, containing various fatty acids optionally enriched with vitamins.

Patent application EP 920 813 describes a method to accelerate the digestion rate of a protein, in particular for the preparation of food supplements for infants.

Patent application EP 126 666 describes food compositions containing carbohydrates, vitamins and mineral salts, a lipid fraction and a protein fraction comprising 3 types of minipeptides, as protein and energy supplement, in particular for the treatment of gastro-intestinal pathologies.

Patent application FR 2 860 976 describes synergic compositions allowing improved bio-availability of polyunsaturated fatty acids, in particular for the treatment of brain function disorders (memory loss, depression or psychological disorders).

Patent application FR 2 520 232 describes a food diet for liver diseases comprising carbohydrates, lipids, vitamins, trace elements and a special mixture of amino acids.

Patent application FR 2 292 437 describes a food for fish and shellfish containing residual fish products with vegetable oils and vitamin cocktails.

Patent application FR 2 497 068 describes a nutritive composition suitable for feeding via enteral route, comprising sources of carbohydrates, proteins, lipids, vitamins etc. to promote feeding by direct injection of food through the gastro-intestinal route via a tube.

Patent application FR 2 691 616 describes nutritional biovectors rich in micronutrients, particularly comprising fatty acids and mixtures of vitamins so as to form pentagonal structures.

Patent application FR 2 706 255 describes food compositions low in polyamines, which can be used in particular as anti-cancer or analgesic agents, or to stimulate immune defenses or further to reduce appetite.

U.S. Pat. No. 6,403,142 describes a hypoallergenic food composition for animals containing hydrolysed proteins. Food compositions for animals are well known, and particularly contain carbohydrates, lipids and vitamins.

U.S. Pat. No. 5,455,235 describes a food composition containing lactosucrose to inhibit the formation of putrefying products in the intestines. These food compositions contain conventional mixes of lipids, proteins and vitamins.

Patent application US 2005/037065 also describes a food composition containing the usual ingredients of this type of composition. The aim of this patent in particular is to associate essential fatty acids with iron as food supplement for pregnant women. The compositions may particularly contain linoleic and linolenic acids to a weight proportion of 1/1, vitamin E and vitamin B9.

Patent GB 806 714 describes an improvement in the stability of pharmaceutical compositions containing vitamin B12, optionally with other vitamins and mixtures of fatty acids.

Patent application EP 1 302 115 describes a nutritional composition containing cystathionine to promote cysteine supply.

Patent application EP 689 834 describes a food supplement containing delayed-release vitamins and minerals.

Patent application US 2002/0032234 describes a food supplement for women before and during breast-feeding. This supplement contains linoleic acid and/or linolenic acid. Compositions containing linoleic and linolenic acids in particular, to a weight proportion of 1/1, vitamin E and vitamin B9 are described.

Mixtures of oils and vitamins are well known in other areas of the prior art, notably for cosmetic applications. For example, patent application EP 330 583 describes a method to improve the cosmetic appearance of the skin using vitamin cocktails optionally associated with oils to form emulsions for topical application.

Therefore, in the prior art very numerous compositions are known which particularly contain mixtures of fatty acids including linoleic and/or linolenic acids, vegetable oils and vitamin cocktails.

However, these compositions are not identified for a particular use with respect to the progression of Alzheimer's disease. This lack of indication for the disease is also due to the choice of fatty acids and oils used in these compositions.

The family of polyunsaturated fatty acids effectively comprises numerous sub-families (omega 3, 6 and 9) and numerous compounds for each one. These different compounds, depending on their chemical structures, have different physiological and pharmacological functions. For example DHA is involved in the synthesis chain of leukotrienes and prostaglandins, by reducing their synthesis, which leads to an anti-inflammatory effect. In different manner, linolenic and linoleic acids insert themselves in cell membranes thereby promoting cell communication and the synthesis of some neurotransmitters. It can be clearly seen that the action observed depends on the type of fatty acid.

The subject-matter of the invention is a specific combination of fatty acids and vitamins which, with use, allows improved slowing of the progression of the disease whilst preserving or maintaining patients' quality of life.

The composition of the invention contains:
a vegetable oil containing at least one linoleic acid and at least one linolenic acid;
vitamin E; and
vitamin B9 (or folates);
   the weight ratio of linoleic acid to linolenic acid lying between 3 and 5, preferably around 4.

According to one preferred embodiment of the invention, the vegetable oil containing at least one linoleic acid and at least one linolenic acid is walnut oil.

The composition of the invention may have different forms depending on the manner it is to be administered.

It may be a concentrate, consisting essentially of the three above-defined components.

It may be a composition of pharmaceutical or nutraceutical type, in which the three components are associated with an acceptable vehicle, more particularly for oral administering, in forms such as tablets, capsules, powders, granules and oral solutions or suspensions.

When a solid composition in tablet form is prepared, the association of the three components according to the invention is mixed with a vehicle such as gelatin, starch, lactose, magnesium stearate, talc, gum arabica or similar. The tablets may be coated with sucrose or other suitable matter, or they may be treated so that they have extended or delayed release and continuously release a predetermined quantity of the association.

A capsule preparation is obtained by mixing the association of the three components according to the invention with a dilutant and pouring the mixture obtained into soft or hard capsules.

A preparation in syrup or elixir form may contain the association of the three components according to the invention together with a sweetener, an antiseptic, and a taste enhancing agent and suitable colouring agent.

Powders or water-dispersible granules may contain the association of the three components according to the invention in a mixture with dispersion agents or wetting agents, or suspending agents, also with taste enhancers or sweeteners.

The composition may of food type in which the association of the three components according to the invention is integrated in a usual food composition, such as cereal bars for example, drinks, prepared meals, cured meats or any type of food preparation that is usually consumed.

The quantity of each of the three constituents of the composition according to the invention depends upon the method of administering (1, 2 or several times a day) and the daily quantity administered. In the composition of the invention, the daily quantity of linoleic acid given to patients lies between 100 and 500 mg, whereas the daily quantity of linolenic acid ranges from 50 to 200 mg.

Preferably, the daily quantity of linoleic acid totals 360 mg, whereas for linolenic acid it is 90 mg.

Regarding vitamin E, the daily quantity in the composition of the invention lies between 5 and 2000 mg. Preferably, the chosen daily quantity of vitamin E is 10 mg.

Finally, further according to the invention, the daily quantity of vitamin B9 must lie between 100 and 500 μg, and is preferably 200 μg.

The vitamins used in the composition of the invention may be vitamins derived from natural extracts or products of chemical synthesis.

The invention also concerns the use of the above composition to prepare a medicinal product allowing delayed progression of Alzheimer's disease or the prevention of its onset.

The use of a vegetable oil containing at least one linoleic acid and at least one linolenic acid has shown its efficacy in animal experiments and in a human clinical trial on Alzheimer's disease. The ratio of acids has been optimised by determining the fatty acid profile and the cholesterol content of the cortex and nerve endings of these structures. This ratio optimises the enzymatic kinetics of fatty acid utilisation, particularly enabling optimal, maximal conversion into very long chain polyunsaturated fatty acids.

Evaluations have been performed by neurologists and psychologists. The main criterion is the statistically significant improvement in the following chief clinical signs: orientation in space, cooperation (with family, relatives, etc.), mood, organization, short and long-term memory, sleep disorders, alertness and attention throughout the day, hallucinations, expression (can the patient express him or herself clearly and transmit ideas).

The combination of these acids is therefore used in the composition of the invention to improve the quality of life of patients.

The two vitamins are chiefly used as protection for the patient. Vitamin E contributes towards neutralizing the free radicals which may accumulate in the body's fatty tissue, and it plays an essential role in the protection of the cell membrane.

Also, during the progression of Alzheimer's disease, vitamin E deficiency in the brain is observed. The use of this substance therefore appears to be indicated to slow the progression of the disease. Additionally it has been noted that the absorption of vitamin-rich foods, vitamin E in particular, strongly correlates with a reduction in the risk of onset of the disease, especially in smokers. The anti-oxidant properties of vitamin E appear to be the reason for this protective effect for the neurones.

Vitamin B9 or folates are involved in the metabolism of homocysteine. A deficiency in these micronutrients leads to accumulation of homocysteine in the plasma, an excessive level inducing lesions of the vascular endothelium. This cytotoxic effect of homocysteine leads to vascular pathologies, especially cerebral pathologies.

Low concentrations of vitamin B9 or folates also disturb the metabolism of myelin, the hypoprotein which ensures the conduction of nerve impulses, of neurotransmitters and membrane phospholipids which are all involved in proper brain functioning.

### EXAMPLE OF COMPOSITIONS

One first composition for a food supplement according to the invention is prepared with the following components:

| | | |
|---|---|---|
| Linoleic acid | 360 | mg |
| Linolenic acid | 90 | mg |
| Vitamin E | 10 | mg |
| Folic acid | 200 | μg |

Another formula can be prepared with the following ingredients

| | | |
|---|---|---|
| Juglans regia | 322.5 | mg |
| (walnut oil) | | |
| Vitamin E | 5 | mg |
| Folic acid | 100 | μg |

The food supplements of the invention can be formulated in capsule form. The capsules, prepared following standard techniques in the pharmaceutical industry, may contain the following constituents: modified starch, glycerol, hydrogenated soy oil, carraghaneen, titanium dioxide, soy lecithin, disodium phosphate.

Recommended use: One or two capsules to be taken per day, at mealtimes with a glass of water for at least 3 months, the course to be repeated as often as necessary.

1. Composition comprising:
a vegetable oil containing at least one linoleic acid and at least one linolenic acid;
vitamin E; and
vitamin B9 (or folates);
wherein the linoleic acid/linolenic acid weight ratio is lying between 3 and 5.

2. The composition of claim 1, wherein the linoleic acid/linolenic acid weight ratio is about 4.

3. The composition of claim 1, wherein the vegetable oil containing at least one linoleic acid and at least one linolenic acid is walnut oil.

4. The composition of claim 1, wherein the quantity of linoleic and linolenic acids is suitable to allow daily administering of between 100 and 500 mg of linoleic acid and between 50 and 200 mg of linolenic acid.

5. The composition of claim 1, wherein the quantity of vitamin E is suitable to allow the daily administering of between 5 and 2000 mg of vitamin E.

6. The composition of claim 1, wherein the quantity of vitamin B9 is suitable to allow the daily administering of between 100 and 500 μg of vitamin B9.

7. The composition of claim 1, wherein it is in a concentrate form.

8. Pharmaceutical composition comprising a composition according to claim 1 and a pharmaceutically acceptable vehicle.

9. Food composition comprising a composition according to claim 1.

10. (canceled)
